(19) United States
(12) Patent Application Publication
(10) Pub. No.: US 2016/0078189 A1
(43) Pub. Date: Mar. 17, 2016

(54) Sedation system and method providing enhanced safety

(71) Applicant: Randall S. Hickle, Lubbock, TX (US)

(72) Inventor: Randall S. Hickle, Lubbock, TX (US)

(73) Assignee: Scott Laboratories, Inc., Lubbock, TX (US)

(21) Appl. No.: 14/756,326

(22) Filed: Aug. 27, 2015

Related U.S. Application Data
(60) Provisional application No. 62/071,175, filed on Sep. 16, 2014.

(51) Int. Cl. G06F 19/00; A61M 5/172; A61M 5/168; A61M 5/142

(52) U.S. Cl. CPC G06F 19/3406; A61M 5/142; A61M 5/1723; A61M 5/16804; G06F 19/3456; A61M 2005/14208

(57) ABSTRACT

A computer assisted sedation system and methods for use by a non-anesthetist procedural physician comprising an electronic controller for a drug infusion system for infusing a sedative, analgesic and/or amnestic drug to a patient having data monitors for determining the patient' physiological data and transmitting same to the controller for determining whether the patient's physiological condition is normal and if not, for signaling an alarm and providing instructions to a non-anesthesiologiest procedural physician to return the patient to a normal condition.

### CROSS REFERENCE TO RELATED APPLICATIONS

This application is based upon and claims priority and other benefits of Provisional Application No. 62/071,175 entitled A SEDATION SYSTEM AND METHOD PROVIDING ENHANCED SAFETY filed Sep. 16, 2014.

### BACKGROUND OF INVENTION

The inventions of this application are related to and comprise enhancements that facilitate the safe and effective administration, of analgesia, sedation and/or amnestic drugs by a computer assisted personalized sedation system to a patient during certain medical and/or surgical procedures by a non-anesthetist, procedural physician. Such provides substantial safety benefits while avoiding the cost of the services of an anesthetist and the high cost of the operating room. Some systems and methods enhanced by these inventions include the disclosures of: U.S. Pat. No. 6,807,965 entitled APPARATUS AND METHOD OF PROVIDING A CONSCIOUS PATIENT RELIEF FROM PAIN AND ANXIETY ASSOCIATED WITH MEDICAL OR SURGICAL PROCEDURES granted Oct. 26, 2004; U.S. Pat. No. 7,229,430 entitled APPARATUSES AND METHODS FOR TITRATING DRUG DELIVERY issued Jun. 12, 2007; U.S. Pat. No. 8,567,393 entitled USER INTERFACE FOR SEDATION AND ANALGESIC DELIVERY SYSTEMS AND METHODS issued Oct. 29, 2013; and U.S. Patent Publication No. 2003/0040700 entitled APPARATUSES AND METHODS FOR PROVIDING IV INFUSION ADMINISTRATION, published Feb. 27, 2003. Pursuant to the provisions of 37 C.F.R. 1.57, the disclosures of these three patents and the publication are incorporated by reference into this application as if fully set forth herein.

In as much as the sedative drug propofol sold under the trademark, Diprivan®, by AstraZeneca is known to cause ventilation suppression and obstruction under certain circumstances, it is labeled by the manufacturer for use only by anesthesiologists. However, the above patents and the publication included numerous safety features to permit the procedural physician to administer propofol and to advise the procedural physician of the onset or occurrence of any adverse condition of the patient relating to the patient's oxygen saturation levels, carbon dioxide levels, heart rate, blood pressure, ECG, level of responsiveness, etc. Too, some embodiments were provided with drug infusion algorithms that were interrupted to reduce or stop the intravenous drug delivery in the event of detection of an adverse patient condition. These interruptions, modifications, fast dissolution of a drug such as propofol, and physician control were believed sufficient to meet needed safety requirements. However, the U.S. Food and Drug Administration initially disagreed and in February of 2010, the FDA issued a non-approval letter of Applicant's sedation system based on its conclusions that propofol was sold with a label that reads “should be administered only by persons trained in the administration of general anesthesia” (anesthesiologists) and that the study data demonstrated that SEDASYS System had been associated with an increased incidence of deeper-than-intended sedation, including episodes of “general anesthesia.” Based on these considerations, the FDA concluded that there was not a reasonable assurance of safety of the subject device when used by non-anesthetist.

After further review, the FDA in May of 2013 granted premarket approval. However, to provide further assurances of safety, Applicant, the inventor, has continuously sought to provide additional procedures and alarms for enhanced safety of the system and associated methods. Such enhanced methods and systems are the subjects of this application.

A primary concern for safety was the actions to be taken by the non-anesthesiologist in the event of an adverse patient condition such as low oxygen saturation, excessive carbon dioxide levels, bradycardia, irregular heart rates, etc. The initial approach of the American Society of Anesthesiologists in their suggestions of an appropriate standard of care was to terminate infusion of the drug propofol. This approach appeared obvious in that the drug propofol is known to cause suppression and/or obstruction of a patient's ventilation and in view of the fact that the drug propofol is a short acting drug that has a very short decrement time in the body. Thus, termination of infusion and decay of the drug in the body appeared to be a quick and appropriate response. Another concern initially noted by the FDA was the stated concern that the level of sedation could result in a “general anesthesia”—a condition that certified anesthesiologists are trained and competent to handle.

### SUMMARY OF THE INVENTIONS

The inventions of this application comprise safety enhancements to computer assisted personal sedation system and methods of infusion. They include the addition of alarms and incorporation of specific procedures into the computer assisted personal sedation system. That system, as previously known, was provided with a drug delivery unit for delivering the drugs to the patient, patient physiological monitors for ECG, oxygen saturation, carbon dioxide levels, etc., and an electronic controller that initiates an algorithm for delivery of the drug and that receives data from the physiological monitors to provide alarms in the event of detection of an adverse physiological condition of the patient. The system also included a user interface connected to the monitor to display the physiological conditions, the drug infusion, and provide certain alarms. Some embodiments of the inventions of the present application facilitate identification of specific problems and provide specific, appropriate directions to the non-anesthesiologist of actions to be taken in the event of an alarm resulting from apparent patient adverse conditions. In addition, in some embodiments, the drug infusion algorithm may be modified to insure that an effect site concentration in the patient is less than a specified amount, e.g., 8 mcg/ml—an amount that is the limit above which the condition of “general anesthesia” may be expected to result.

Accordingly, the objects of the present invention are to provide, among other things, an enhanced safety system and methods for enabling a non-anesthetist to perform a medical and/or surgical procedure by using computer assisted system having an electronic controller for a drug delivery system to infuse the drugs into the patient, physiological monitors for communicating the physiological condition of the patient to the controller to determine if the patient condition is abnormal, and if so, to provide appropriate alarms and directions for correction of any abnormal procedure. A preferred embodiment of the inventions are reflected in the following drawings and specification.

### DESCRIPTION OF THE DRAWINGS FIG. 1 is a block diagram of a preferred embodiment of the computer assisted personal sedation system and its components; and

FIG. 2 is an illustration of a preferred embodiment of user interface that illustrates a primary safety component of the system.

### DETAILED DESCRIPTION OF THE INVENTIONS

As depicted in FIG. 1, a computer assisted sedation system may be connected to a conscious, non-intubated patient in need of analgesia, sedation and/or amnesia during a medical and/or surgical procedure. The system may include an electronic controller 2 for controlling infusion of an intravenous drug to a patient 6 by a drug delivery unit 4. Before and during the procedure, the patient's physiological conditions are obtained by monitors 8 whose data is transmitted to the controller 2 to reflect the patient's physiological condition. In the event the patient's physiological condition becomes abnormal, the controller 2 issues alarms and directives to a user 12 through a user interface 14. A power supply 10 may provide electrical power to controller 2 and other electronic components of the system.

Further explaining these components, the controller 2 may be comprised of a programmable computer processor, e.g., a microchip such as XK21E and its successors provided by Texas Instruments or others who provide substitutes that accomplish the functions described herein, together with electronic memory elements 16 that are well known and commercially available. Memory 16 may be part of controller 2 or separate from but in communication with controller 2. This controller 2 or its various substitute units may be placed on a logic board or other suitable computing device. The memory 16 is used to store a safety data set which reflects normal and/or abnormal physiological conditions of a patient. In addition, the memory 16 contains an algorithm for controlling the rate of the drug to be infused by the drug delivery unit 4. This algorithms permits the input of variables related to the patient weight and sex and other factors that may be necessary to ascertain the most desirable and effective rate of infusion.

Consequently, the controller 2 must be capable of receiving data from physiological monitors 8, comparing that data to the safety data set of the memory 16, and providing output to a user interface 14 to communicate the condition of the patient 6 to the user 12. Other functions of the controller 2 may include the receipt of instructions and data input to modify the drug delivery algorithm as needed for the each patient through the patient interface 14, to start the drug delivery, to deliver drugs in accord with the algorithm of the memory 16 or, in response to instructions from the user 12, to override and modify the drug infusion rate set by the delivery algorithm or the user 12 or to terminate that delivery.

The drug delivery unit 4 is preferably a digitally controlled peristaltic pump which draws drug from a drug vial or other drug source and infuses same through a delivery conduit into the patient's veins. Of course, any suitable drug delivery unit 4 may be used. Various commercial devices having very accurate control of the drug flow are well known and commercially available on the market for this purpose.

Preferably, in some embodiments, the patient monitors 8 may include a pulse oximeter, a capnometer to measure carbon dioxide levels, a blood pressure and heart rate monitor, ECG, etc. Each of these monitors may be attached to the patient and its output may be delivered to the controller 2 for comparison with a safety data set held in memory 16 associated with the controller 2. In the event the data from any of the monitors is outside a safe range, an alarm is sounded and/or displayed on the user interface 14.

Numerous sedative, analgesic and amnestic drugs can be infused. However, the greatest need and cost saving benefits are expected to come from the sedative propofol. It is a very fast on and off drug, permits the physician to quickly commence the procedure after infusion begins, and enables the patient's brain levels at the effect site of the drug to predictably and rapidly decrement following cessation of administration, returning the patient to a nominal baseline or normal condition without adverse effects within a very short period of time, such as an hour, for example. Moreover, it is very effective as a sedative eliminating anxiety. An example of a primary intended use is to perform gastrointestinal colonoscopies and endoscopies.

However, the use of propofol has potential adverse consequences. At some level of infusion and concentration, propofol can cause central suppression of the drive to breathe and/or airway obstruction, both effects having the potential to suppress ventilation of sufficient magnitude to cause patient harm. On belief, this fact has limited use of propofol to certified anesthesiologists and nurse anesthetists with access to ventilation equipment in the operating room. Accordingly, it is very important to ascertain the maximum level of propofol to be infused, to avoid exceeding that level, and, in the event that ventilatory suppression should occur, the procedural physician who is relying on the computer assisted sedation system, rather than an anesthetist, must be able to manage the patient's condition to prevent harm. Some embodiments of this invention may include an appropriate, novel response to adverse conditions and may provide instructions for proper standards of care to manage the patient out of the potentially adverse incident. As shown in the drawings, such improvements may be integrated into the sedation system. That integration may take place with the user interface 14 as described herein.

The user interface unit 14 preferably comprises a touch screen with a GUI (Graphical User Interface) that facilitates transition from screen display to screen display, each providing additional information as desired, such information including, for example, graphs of the drug rate of infusion, the quantity of infusion at any point in time, and the patient's physiological conditions such as heart rate, blood pressure, oxygen saturation, carbon dioxide levels, ventilation cycle, etc. Data for these graphs, provided by the monitors 8 identified above, is transmitted to the controller 2, which in turn provides the appropriate display to the user interface 14. In addition, the display may be provided with control buttons for certain operations as well as a keypad or other I/O device. For example, the control buttons may be used to reduce or terminate infusion of the drug, and the key pad can be used to insert the patient's weight, age, sex, or other data into the controller 2 for input to the infusion algorithm. Of course, any suitable interface and I/O devices may be used for user interface 14, depending on the particular application.

In some embodiments pertinent to this invention, the user interface 14 is used to specifically guide the non-anesthetist, procedural physician 12 through potential problems that might be incurred with the drug. A primary potential problem is inadequate respiratory ventilation, caused either by suppression of the drive to breathe and/or mechanical airway obstruction, which may be caused by the infusion of the drug propofol.

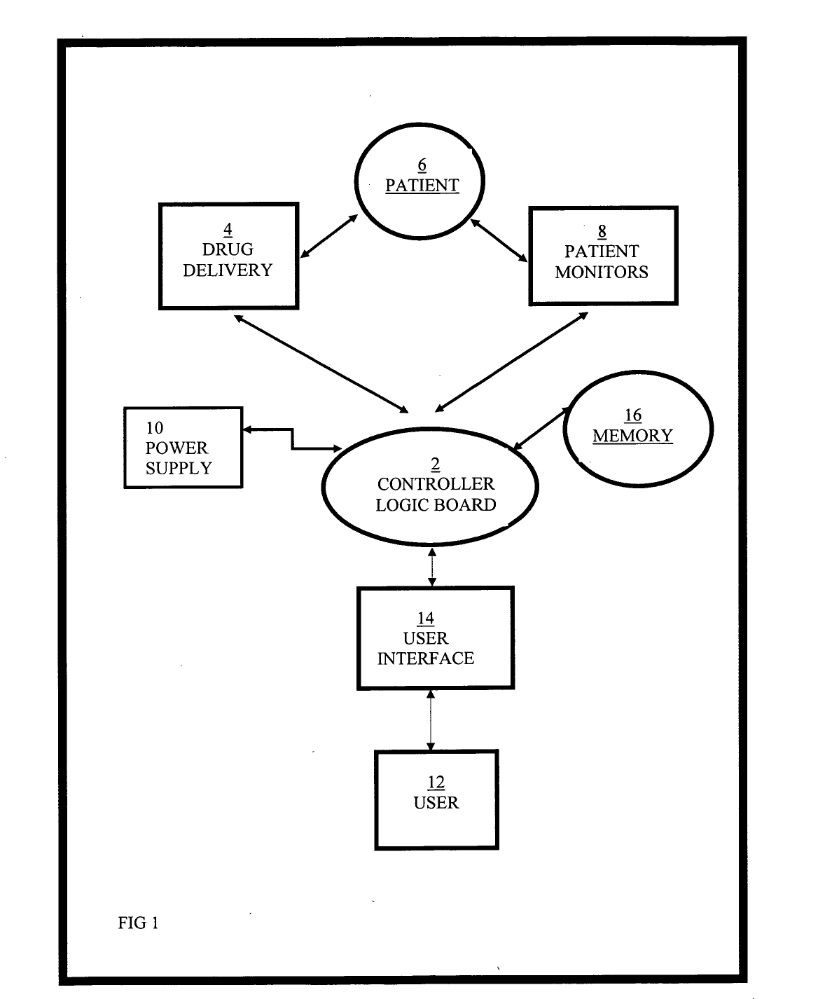

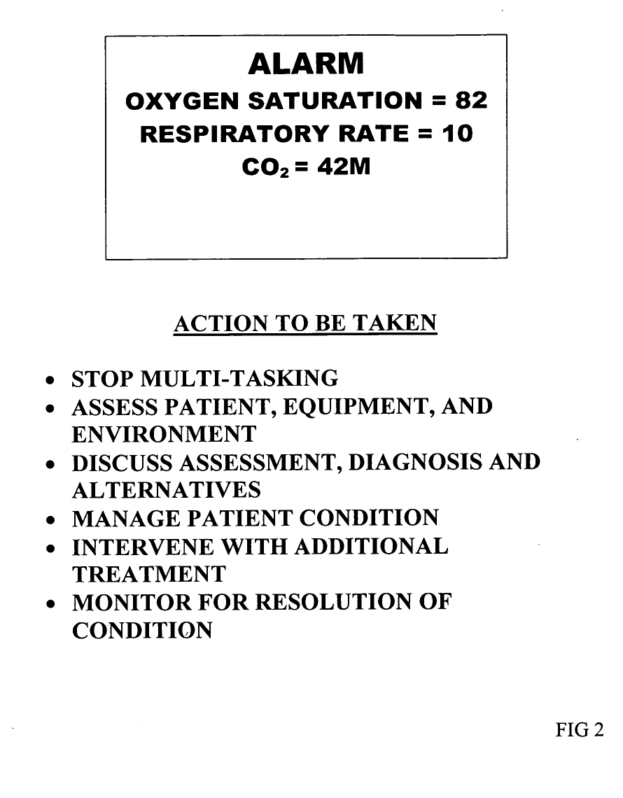

To manage this problem, the Controller 2 is configured to compile multiple monitored signals from monitors 8 and place them on the screen of user interface 14. For example, information pertinent to detection of ventilation suppression may be available from the monitors in three forms: 1) oxygen saturation; 2) respiratory rate; and 3) carbon dioxide level. Simultaneous display of all three of those sets of data is surprisingly important, even though only one set of such monitored data may be outside the data safety set. Such an arrangement and communication of an alarm is depicted in FIG. 2, which displays the Alarm and discloses the three data sets reflecting different potential causes of the possibility of ventilation suppression.

In response to an alarm regarding potentially inadequate respiratory ventilation in a case involving infusion of propofol by a non-anesthesiologist physician, the reaction and obvious recommendation of most anesthesiologists is to establish a guideline which would instruct the first step of immediate reduction or termination of drug infusion. Indeed, prior patents suggest different protocols such as modification or termination of the infusion of the drug propofol. (See for example, U.S. Pat. No. 6,807,965, FIG. 21, and column 30, lines 52 through column 32, lines 29). Such, however, may not be the best in the case of sedation by the procedural physician without the assistance of an anesthesiologist.

As will be demonstrated, the user interface disclosure of FIG. 2 depicting the ALARM, the Data and the prescribed ACTIONS TO BE TAKEN, result in an outcome that is not only contrary to the obvious, expected actions prescribed by guidelines of anesthesiologists, it also describes the preferred, response to resolution of the problem. It is for the non-anesthetist procedural physician to:

STOP multitasking, i.e. stop the medical or surgical procedure and focus on the problem;

ASSESS patient, equipment, and environment;

DISCUSS with other personnel the assessment, diagnosis, and alternatives;

MANAGE the patient condition;

INTERVENE with alterations of drug infusion or other appropriate therapies; then

MONITOR to determine if the diagnosis and treatments are leading the patient toward safe resolution of the alarm condition.

One example in which the preferred guideline disclosed in the invention herein results in actions that are entirely different than previously existing anesthesiologist guidelines and protocol is presented below. In this case, by STOPPING, ASSESSING the patient, equipment, and environment, and DISCUSSING the assessment, diagnosis and alternatives, the procedural physician or nurse assistant would immediately perceive that the oxygen saturation is far below normal, but the respiratory rate and the carbon dioxide tracings are normal, and they would quickly identify that the pulse oximeter has merely slipped from the patient's finger. So, following the protocol above, no change in drug infusion, oxygen supply or carbon dioxide levels is necessary. Upon restoration of the misplaced monitor 8, which is the root cause of the alarm condition, the monitor 8, through the user interface 14, will demonstrate restoration of normal monitored parameters of the patient's condition. Clearly, this procedure is far preferable to the prior “obvious” solution of termination of infusion, which has the very high risk of creating anxiety or pain during the medical procedure.

Another improvement of the system relates to the setting of the maximum drug infusion level for propofol. The infusion level is often referred to as the “Effect Site Concentration” (ESC) and its determination is well known to those in the industry. Such is normally used in conjunction with the infusion concept of Target Controlled Infusion (TCI) which establishes the rate of infusion and other parameters. However, it is believed that the ESC can be calculated or derived for all protocols of drug infusion.

In the inventor' practice, his study of research reports, and his consultations with others, has resulted in the conclusion that for conscious sedation, the preferred upper limit for ESC for “conscious sedation” is 4 mcg/ml. However, additional information suggests that a preferred upper limit for “deep sedation,” in which the painful or repeated stimulation of the patient is required to obtain a response, is an ESC of 6 mcg/ml. Accordingly, another improvement to the existing systems is to continue to recommend 4 mcg/ml as the preferred maximum level and to modify all infusion algorithms to limit infusion to no greater than 6 mcg/ml. The various levels of sedation that lead to “general anesthesia” is fully described by the American Society of Anesthesiologists in a Special Article entitled “Practice Guidelines for Sedation and Analgesia by Non-Anesthesiologists,” which was copyrighted in the year 2002.

Those skilled in the art will appreciate that the inventions disclosed and claimed in this application may be modified in various ways without departing from the disclosure and claims. Accordingly, the following claims are intended to cover each of those modifications and derivations from the use of my disclosure. The embodiments described herein are some examples of the current invention. Various modifications and changes of the current invention will be apparent to persons of ordinary skill in the art. Among other things, any feature described for one embodiment may be used in any other embodiment. The scope of the invention is defined by the attached claims and other claims to be drawn to this invention, and is not limited to the specific examples described herein.

I claim

1. A computer assisted personal sedation system with enhanced safety features to enable non-anesthetist personnel, including a procedural physician, to provide safe and effective sedation, amnesia and/or analgesia without inducing deep sedation and/or general anesthesia in a patient during a medical and/or surgical procedure, said system comprising:
a. an electronic controlled drug delivery system for delivering an intravenous sedative, amnestic and/or analgesic drug to a patient during said medical and/or surgical procedure;
b. physiological monitors for connection to said patient for determining and providing physiological data pertaining to the patient's physiological condition during said medical and/or surgical procedure;
c. a safe and effective data set containing safe and/or unsafe parameters of monitored data stored in a digital electronic memory;
d. a digital electronic controller accessible to said parameters and in communication with said monitors for receiving said physiological data and comparing same to said parameters, said controller also in communication with said drug delivery system for controlling the rate and quantity of infusion of said drug into said patient; and
e. a user interface in communication with said controller, said user interface configured for receiving and displaying said physiological data and providing an alarm in the event said patient's physiological condition becomes unsafe;
f. said user interface adapted to display at least three parameters relating to ventilation and a protocol for managing the unsafe condition.

2. A system as recited in claim 1 in which said protocol is stated on the user interface at the time of unsafe condition and identifies the following steps to be taken:
STOP multi-tasking;
ASSESS patient, equipment, and environment;
DISCUSS assessment, diagnosis, and alternatives;
MANAGE patient condition;
INTERVENE with additional treatment;
MONITOR for resolution of condition.

3. The system of claim 1 wherein said three parameters comprise oxygen saturation, respiratory rate, and carbon dioxide level.

4. The system of claim 1 wherein said drug comprises propofol and wherein said parameters comprise an upper limit of ESC for “conscious sedation” of 4 mcg/ml.

5. The system of claim 4 wherein said parameters further comprise an upper limit of ESC for “deep sedation” of no greater than 6 mcg/ml.

6. A method for providing computer assisted personal sedation with enhanced safety features to enable non-anesthetist personnel, including a procedural physician, to provide safe and effective sedation, amnesia and/or analgesia without inducing deep sedation and/or general anesthesia in a patient during a medical and/or surgical procedure, said method comprising the steps of:
a. intravenously infusing a sedative, amnestic and/or analgesic drug by an electronically controlled drug delivery system to a patient during said medical and/or surgical procedure;
b. monitoring physiological data of said patient and transmitting said data to an electronic controller for comparison with safe and or unsafe data pertaining to said physiological level and for providing in alarm in the event said physiological data of said patient becomes unsafe;
c. transmitting a signal to a user interface in the event said physiological data becomes unsafe and providing all physiological data relevant to said unsafe condition to enable said non-anesthesiologist to assess the patient, the equipment and environment, to manage the unsafe condition and intervene with additional treatment.

7. A method as recited in claim 6 in which said physiological data includes data pertaining to the patient's carbon dioxide level, oxygen level and respiratory rate in the event that the alarm is related to the patient's ventilation.

8. A method as recited in claim 6 in which the maximum infusion rate is limited to 4 mcg/ml.

9. A method as recited in claim 6 in which the maximum infusion rate is limited to 6 mcg/ml.

10. A method as recited in claim 6 in which the following protocol steps are displayed on the user interface in the event of an unsafe condition:
STOP multi-tasking;
ASSESS patient, equipment, and environment;
DISCUSS assessment, diagnosis, and alternatives;
MANAGE patient condition;
INTERVENE with additional treatment;
MONITOR for resolution of condition.

11. A computer assisted personal sedation system having enhanced safety features to enable non-anesthetist personnel, including a procedural physician, to provide safe and effective sedation, amnesia and/or analgesia without inducing deep sedation and/or general anesthesia in a patient during a medical and/or surgical procedure, said system comprising:
a. an electronic controlled drug delivery system for delivering an intravenous sedative, amnestic and/or analgesic drug to a patient during said medical and/or surgical procedure;
b. physiological monitors for connection to said patient for determining and providing physiological data pertaining to the patient's physiological condition during said medical and/or surgical procedure;
c. a memory having data reflecting safe and/or unsafe physiological data for said patient.
d. a controller for receiving said physiological data from said monitors and for comparing said received data to said safe and/or unsafe physiological data and for
e. a safe and effective data set containing safe and/or unsafe parameters of monitored data stored in a digital electronic memory;
f. a user interface for displaying a procedural protocol of the proper steps of a standard of care to be taken in the event the condition of the patient becomes unsafe.
MONITOR for resolution of condition.
